(19) United States
(12) Patent Application Publication
(10) Pub. No.: US 2025/0229093 A1
(43) Pub. Date: Jul. 17, 2025

(54) BACKFILL FEATURE FOR IMPLANTABLE MEDICAL DEVICE

(71) Applicant: Greatbatch Ltd., Clarence, NY (US)

(72) Inventors: Ignacio Agustin Armesto, Montevideo (UY); Luis Daniel Villamil, Montevideo (UY); Carlos Santana, Montevideo (UY)

(21) Appl. No.: 19/014,348

(22) Filed: Jan. 9, 2025

Related U.S. Application Data
(60) Provisional application No. 63/619,795, filed on Jan. 11, 2024.
(60) Provisional application No. 63/553,682, filed on Feb. 15, 2024.

(51) Int. Cl. A61N 1/375; A61B 90/50

(52) U.S. Cl. CPC A61N 1/3754; A61B 90/50

(57) ABSTRACT

In various examples, an implantable medical device includes an enclosure including an enclosure wall. A feedthrough is sealingly disposed within an opening of the enclosure wall. A backfill feature includes a backfilling channel open to an exterior of the implantable medical device. A welding protection channel is fluidly coupled to the backfilling channel and extends from the backfilling channel to an interior of the enclosure. The backfilling channel and the welding protection channel fluidly couple the exterior of the implantable medical device and the interior of the enclosure for fluid backfilling of the interior of the enclosure. The backfilling channel is configured to be sealed with welding of the backfilling channel after fluid backfilling of the interior of the enclosure. The welding protection channel is configured to catch metallic projections from the welding of the backfilling channel to inhibit the metallic projections from entering the interior of the enclosure.

### CROSS REFERENCE TO RELATED APPLICATIONS

This application claims the benefit of priority to U.S. Provisional Application Ser. No. 63/619,795, filed on Jan. 11, 2024, entitled “FEEDTHROUGH WITH HE BACKFILL WELDING PROTECTION” and U.S. Provisional Application Ser. No. 63/553,682, filed on Feb. 15, 2024, entitled “PRE-ASSEMBLED HEADER WITH GAS BACKFILL FEATURE FOR AIMD,” each of which is incorporated by reference herein in its entirety.

### BACKGROUND

Implantable medical devices, particularly active implantable medical devices, typically have an inert atmosphere inside the hermetic enclosure that houses the electronics to avoid any deterioration or corrosion of the electrical components in the long term. One of the processes extensively used to ensure that a hermetic portion of the implantable medical device is fully backfilled with an inert gas, and thereby fulfilling the industrial practice of having an inert atmosphere, includes a small hole in the device enclosure. This hole is typically incorporated into the enclosure or the lid of the device (hermetic encapsulation) to perform a fluid (typically gas, such as helium, argon, nitrogen, or the like or a combination thereof) backfilling process before the hole is hermetically welded shut. To close the hole during the fluid backfilling process, the hole is typically laser seal welded. This process forms a pool of liquid metal, that then solidifies and closes the hole. But, in some conditions, liquid metallic projections can result from the welding process that, in some situations, can migrate through and down the hole, which, if a metallic projection reaches an interior of the implantable medical device, can damage the internal components of the implantable medical device, for instance, by creating a short-circuit within the electronic componentry. To reduce this possible risk, usually, a metallic strip or band is welded or machined to the back of the small hole of the implantable medical device to inhibit such metallic projections from coming in contact with the inner components of the implantable medical device. Typically, a titanium band or strip is used to protect the inner electrical components from any metallic projections that may occur during the seal welding. The addition of this band adds a component and a welding process to the manufacturing of the device. Also, the band or strip can be deformed during the manufacturing stages and therefore become unable to adequately protect the inner electrical components of the device.

Implantable medical devices, particularly active implantable medical devices, typically have a polymer (such as epoxy, for instance) header as part of the device. The header typically includes one or more lead cavities and/or one or more antennas within the header. Typically, an epoxy header is formed using a casting process, the header being formed over a hermetic enclosure assembly of the implantable medical device. The hermetic enclosure assembly typically includes electronics of the implantable medical device inside a metallic enclosure, which is laser welded to secure the hermetic enclosure assembly with the inert gas atmosphere inside of the implantable medical device.

This epoxy casting process is usually the assembly stage with the lowest yield, and failures in this process could signify having to discard the implantable medical device. Also, after the epoxy casting process, most of the components are already assembled and the manufacturing stages are already performed, which highly increases the cost of discarding a device at this stage. Additionally, the epoxy-casting process is usually a bottleneck in the manufacturing stages of the implantable medical device because it is typically a manual and time-consuming process. Considering the bad yield, typically, a significant number of units (perhaps about 40-50%) require some re-processing, significantly increasing manufacturing time.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present inventive subject matter can be used to reduce a number of components needed in implantable medical devices, thereby reducing the complexity of the assembly, reducing assembly times, and allowing for a smaller device by reducing the need for a weld protection band to protect inner electrical components during welding of the device. In various examples, the present inventive subject matter is advantageous in that it provides a machined backfilling hole that, in some examples of the present inventive subject matter, can be better controlled in dimensions and tolerances. Moreover, in some examples, the present inventive subject matter provides for a header assembly to be made in parallel with the rest of the device assembly, thereby reducing the assembly times. Also, by casting the header assembly without the hermetic enclosure assembly, the subassembly volume for each unit is reduced and more units can be cast on a single batch, and the curing schedule can be optimized without any limitation/constraint caused by the electronic components (for instance, temperature limitations/constraints). In this way, in various examples, the present inventive subject matter is advantageous in that it provides a significant cost reduction or optimization of components and resources and a process suitable for automatization. To better illustrate the devices described herein, a non-limiting list of examples is provided here:

Example 1 can include subject matter that can include an implantable medical device. An enclosure includes an enclosure wall. The enclosure wall surrounds and defines an interior within the enclosure. The enclosure includes an opening within the enclosure wall. A feedthrough is sealingly disposed within the opening of the enclosure wall. A backfill feature includes a backfilling channel open to an exterior of the implantable medical device. A welding protection channel is fluidly coupled to the backfilling channel and extends from the backfilling channel to the interior of the enclosure. The welding protection channel is disposed at an angle to the backfilling channel, wherein the backfilling channel and the welding protection channel fluidly couple the exterior of the implantable medical device and the interior of the enclosure to allow for fluid backfilling of the interior of the enclosure. The backfilling channel is configured to be sealed with welding of the backfilling channel after fluid backfilling of the interior of the enclosure. The welding protection channel is configured to catch metallic projections from the welding of the backfilling channel to inhibit the metallic projections from entering the interior of the enclosure.

In Example 2, the subject matter of Example 1 is optionally configured such that the backfill feature is disposed within the feedthrough.

In Example 3, the subject matter of Example 2 is optionally configured such that the backfill feature is integrated within a flange of the feedthrough.

In Example 4, the subject matter of any one of Examples 1-3 is optionally configured such that the backfill feature is disposed within the enclosure wall.

In Example 5, the subject matter of any one of Examples 1-4 is optionally configured such that, with the feedthrough sealed within the opening of the enclosure and the backfilling channel sealed, the interior of the enclosure includes a hermetic environment.

In Example 6, the subject matter of any one of Examples 1-5 optionally includes a header attached to the enclosure, wherein the backfill feature is accessible from the exterior of the implantable medical device with the header attached to the enclosure.

In Example 7, the subject matter of any one of Examples 1-6 optionally includes a header attached to the enclosure, wherein the backfill feature is covered by the header with the header attached to the enclosure.

In Example 8, the subject matter of any one of Examples 1-7 is optionally configured such that the backfilling channel includes a cross section that is smaller than a cross section of the welding protection channel.

In Example 9, the subject matter of any one of Examples 1-8 is optionally configured such that the backfilling channel is circular in cross section and the welding protection channel is circular in cross section. A diameter of the backfilling channel is smaller than a diameter of the welding protection channel.

In Example 10, the subject matter of any one of Examples 1-9 is optionally configured such that the angle between the backfilling channel and the welding protection channel is less than 180 degrees and greater than zero degrees.

In Example 11, the subject matter of any one of Examples 1-10 is optionally configured such that the angle between the backfilling channel and the welding protection channel is about 90 degrees.

Example 12 can include, or can optionally be combined with any one of Examples 1-11 to include subject matter that can include an implantable medical device. An enclosure includes an enclosure wall. The enclosure wall surrounds and defines an interior within the enclosure. The enclosure includes an opening within the enclosure wall. A feedthrough is sealingly disposed within the opening of the enclosure wall. A backfill feature includes a backfilling channel open to an exterior of the implantable medical device. A welding protection channel is fluidly coupled to the backfilling channel and extends from the backfilling channel to the interior of the enclosure. The welding protection channel is disposed at an angle to the backfilling channel. The angle being less than 135 degrees and greater than 45 degrees, wherein the backfilling channel includes a cross section that is smaller than a cross section of the welding protection channel. The backfilling channel and the welding protection channel fluidly couple the exterior of the implantable medical device and the interior of the enclosure to allow for fluid backfilling of the interior of the enclosure. The backfilling channel is configured to be sealed with welding of the backfilling channel after fluid backfilling of the interior of the enclosure. The welding protection channel is configured to catch metallic projections from the welding of the backfilling channel to inhibit the metallic projections from entering the interior of the enclosure.

In Example 13, the subject matter of Example 12 is optionally configured such that the backfill feature is disposed within the feedthrough.

In Example 14, the subject matter of Example 13 is optionally configured such that the backfill feature is integrated within a flange of the feedthrough.

In Example 15, the subject matter of any one of Examples 12-14 is optionally configured such that the backfill feature is disposed within the enclosure wall.

In Example 16, the subject matter of any one of Examples 12-15 is optionally configured such that, with the feedthrough sealed within the opening of the enclosure and the backfilling channel sealed, the interior of the enclosure includes a hermetic environment.

In Example 17, the subject matter of any one of Examples 12-16 optionally includes a header attached to the enclosure, wherein the backfill feature is accessible from the exterior of the implantable medical device with the header attached to the enclosure.

In Example 18, the subject matter of any one of Examples 12-17 optionally includes a header attached to the enclosure, wherein the backfill feature is covered by the header with the header attached to the enclosure.

In Example 19, the subject matter of any one of Examples 12-18 is optionally configured such that the backfilling channel is circular in cross section and the welding protection channel is circular in cross section. A diameter of the backfilling channel is smaller than a diameter of the welding protection channel.

In Example 20, the subject matter of any one of Examples 12-19 is optionally configured such that the angle between the backfilling channel and the welding protection channel is about 90 degrees.

### BRIEF DESCRIPTION OF THE DRAWINGS

FIG. 1 is a perspective view of an implantable medical device including a backfill feature in accordance with at least one example of the invention.

FIG. 2 is an exploded perspective view of the implantable medical device of FIG. 1.

FIG. 3 is a perspective view of a feedthrough of the implantable medical device of FIG. 1, the feedthrough including the backfill feature.

FIG. 4 is a top view of the feedthrough of FIG. 3.

FIG. 5 is a cross-sectional view of the feedthrough of FIG. 4 taken along line 5-5.

FIG. 6 is a close-up cross-sectional perspective view of a portion of the implantable medical device of FIG. 1 including the backfill feature, the implantable medical device having some components removed for clarity in depicting the backfill feature.

FIG. 7 is a perspective view of an implantable medical device including a backfill feature in accordance with at least one example of the invention.

FIG. 8 is an exploded perspective view of the implantable medical device of FIG. 7.

FIG. 9 is a cross-sectional perspective view of the implantable medical device of FIG. 7 depicting the backfill feature.

FIG. 10 is a close-up cross-sectional view of a portion of the implantable medical device of FIG. 7 including the backfill feature, the implantable medical device having some components removed for clarity in depicting the backfill feature.

FIG. 11 is a perspective view of an implantable medical device including a backfill feature in accordance with at least one example of the invention.

FIG. 12 is a perspective view of an enclosure portion of the implantable medical device of FIG. 11, the enclosure portion including the backfill feature.

FIG. 13 is a side view of the enclosure portion of FIG. 12.

FIG. 14 is a close-up cross-sectional view of a portion of the enclosure portion taken along line 14-14 of FIG. 13.

### DETAILED DESCRIPTION

The present inventive subject matter relates generally to an implantable medical device. More specifically, the present inventive subject matter relates to a fluid backfill feature of an implantable medical device. In various examples, the fluid backfill feature provides protection for inner componentry of the implantable medical device during welding of the fluid backfill feature to seal the implantable medical device. In some examples, the fluid backfill feature allows for filling of the implantable medical device after a header has been formed on the implantable medical device.

Conventional systems typically use a weld protection band that is separately attached at an interior side of a backfilling hole to protect the inner electrical components of an implantable medical device during welding operations of the backfilling hole. In some examples, a backfilling hole and weld protection can be incorporated into the device enclosure if the enclosure is machined. In such cases, the cost associated with machining enclosures is relatively high and typically not recommended for mass-production devices. However, when using conventional stamped enclosures, the separate weld protection band or part is needed.

The present inventive subject matter, in some examples, is directed to a backfill feature, including a backfilling hole or channel and a weld protection channel, that is integrated into an enclosure or feedthrough, thereby reducing the number of components and manufacturing stages for the device. In some examples, the backfill feature can be incorporated into a flange of the feedthrough, and, since the feedthrough flange is already a machined component, adding this extra feature does not impact cost or add a new process to the feedthrough itself as a component. In some examples, since the backfill feature of the present inventive subject matter eliminates the need for a weld protection band, extra room within the enclosure for the weld protection band is no longer needed, allowing for a reduction of the device volume. In some examples, the backfill feature of the present inventive subject matter enhances welding performance to the point that high-quality sealed welding can be obtained even when having slight variations in the parts/equipment and/or fixtures set up.

In some examples, weld protection can be formed within a machined enclosure to eliminate the need of any strip, thereby reducing assembly steps for the device and allowing for more repeatable protection during welding. In some examples, the backfill feature with weld protection can be formed within the feedthrough, and, in some instances, within the flange of the feedthrough, thereby eliminating the separate weld protection band and reducing the number of components and manufacturing stages of the device. In some examples, this configuration also can reduce the volume of the device, since the weld band consumes more space than the protection integrated on the feedthrough flange. In some examples, the hole diameter and tolerance can be better controlled, and the hole depth can be deeper compared with a hole in a stamped enclosure. In some examples, the present inventive subject matter can control all these features and, therefore, achieve a better seal welding and enhanced process. Moreover, in some examples, the backfill feature with weld protection disposed within the feedthrough allows for weld protection (without the need for a separate weld band) in devices with stamped enclosures, which are more conventional and better suited for devices with high production than machined enclosures.

A glove-box laser apparatus can be used to close and backfill a device with inert gas without the device needing a backfill hole because all the assembly processes and sealing of the device are performed under a positive inert gas pressure. However, the backfill feature with weld protection in some examples of the present inventive subject matter has several advantages over a glove-box process. In some examples, using the backfill feature allows for assembly of the device in a non-inert atmosphere, thereby simplifying the assembly process. Additionally, in some examples, the use of the backfill feature allows for different gases or other fluids to be used to backfill the device, whereas the glove-box apparatus has several limitations with changing gas or gas mixing and does not allow for the selection of the proper or more appropriate gas, as is needed in each different process.

Conventional systems use an inert gas backfilling hole in the feedthrough flange in a location that is then covered with epoxy once the header is formed. This configuration is not compatible with making a separate header assembly, since, after welding the header assembly to the enclosure, the device needs to be backfilled.

In contrast, in some examples, the backfill feature of the present inventive subject matter can be included within the feedthrough flange in a location that is not covered in epoxy once the header is in place on the device. In some examples, the backfill feature includes a backfilling hole geometry suited to be laser welded without damaging the inner components of the implantable medical device and without the necessity to add a secondary component/process (such as attaching a separate weld band or strip) to protect the inner components of the implantable medical device. In this way, in some examples, the backfill feature being accessible after the header is in place on the implantable medical device allows the header assembly to be made in parallel with the rest of the device when laser welding with direct gas protection and a backfilling process is needed.

Conventional systems are tested for inert gas leakages at a hermetic enclosure assembly level, meaning that the test is performed once the enclosure is welded hermetically with its inner components. On devices with deep-drawn enclosures, the test is performed after welding the feedthrough to the enclosure. On devices with shallow-drawn enclosures, the test is performed after welding the enclosure halves. In both cases, the epoxy and header components are not present during the test, since porous materials (such as, for instance, the silicone lead cavity seals or the silicone septum seals as well as the epoxy) absorb gases that are released during the leakage test and can produce false fails.

The present inventive subject matter, in some examples, defines a method of performing the gas leakage test in the presence of silicone or other porous materials without producing false fails. This would allow for the inert gas backfilling process to be made after adding the epoxy header to the device.

By modifying the feedthrough flange to allow for the backfilling process to be made after the epoxy header is formed, in some examples, the header assembly can be made in parallel to the rest of the device assembly. This can improve the overall manufacturing time of the device, reduce the manufacturing bottlenecks, and significantly reduce the costs associated with failures during the epoxy casting process.

In some conventional systems, pre-molded headers can be used which include a polymer header injected standalone with components to be fit inside defined cavities/features thereafter. Such a design is possible in simple headers with few components. But when a wide variety of components need to be placed in a header, such a pre-molded header is unable to be used. Moreover, the attachment of a pre-molded header to an enclosure is a challenge that has created several recalls in the industry. The epoxy casting of the header used with the backfill features of the present inventive subject matter is significantly more robust and includes a very high adhesion strength to the enclosure.

In some examples, with the header being produced in parallel to the rest of the implantable device, any failure in the epoxy casting process that has the consequence of discarding the header assembly will have a significantly lower cost relative to the same failure on a full device which would require discarding of the entire device. The number of components and manufacturing stages in a header assembly is much lower than on a full device. In this way, in some examples, a significant cost reduction or optimization of components and resources can be achieved.

The purpose of the present inventive subject matter, in some examples, is to reduce the size of an implantable medical device by removing the need for a welding protection component and, in turn, reduce the number of assembly operations by reducing assembly risk and enhancing yield. In some examples, this can be accomplished with a fluid backfilling hole or channel and a welding protection channel integrated into a feedthrough flange. In some examples, the backfill feature of the present inventive subject matter allows for a welding junction (hole shape and depth) that can be better controlled by design. In some examples, the feedthrough flange is already a high-precision machined component, therefore there is no new process necessary to machine the backfill feature.

When a conventional backfilling hole is added to the device enclosure, because the wall thickness of the enclosure is relatively thin, challenges are created with the welding process. In addition, welding protection must be added in order to prevent circuit board damage or other damage to the electronics of the device. Therefore, this kind of conventional design has an extra part and assembly process combined with welding that demands more process controls.

The present invention, in some examples, allows the header to be cast as a sub-assembly process. In this way, in some examples, the full header process (that can be a bottleneck in the assembly line and where major yield issues are present) can be assembled in parallel with the rest of the device. The present inventive subject matter, in some examples, includes various backfill feature configurations that allow manufacturing of the device with a feedthrough flange design suitable for welding in proximity to an epoxy polymer. In some examples, various backfill feature configurations allow for the manufacture of the header assembly in parallel with the enclosure and the electronics therein and the inert gas backfilling process to be performed with the epoxy header formed in place on the device.

Some advantages of the present inventive subject matter include reduced lead times for the full device assembly. Also, significant reduction in cost can be achieved as any yield issue with the conventional process requires discarding the full device (which already has incorporated expensive components like a battery, PCBs, and the like), whereas a yield issue with the implantable medical device of the present inventive subject matter would only require discarding of the header assembly. In some examples, the present inventive subject matter is advantageous in that it provides optimization of temperature during epoxy curing since there are no electronics present during epoxy curing (typically, the curing schedule is constrained by the electronic temperature limitations).

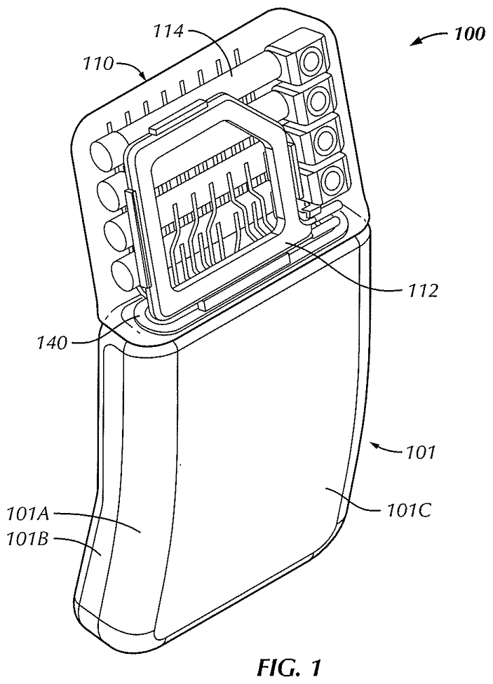

Referring now to the drawings wherein like reference numerals identify similar structural elements or features of the subject invention, there are illustrated in FIGS. 1, 7, and 11 various examples of implantable medical devices 100, 700, 1100, each configured for implantation at a location within a patient. The implantable medical devices 100, 700, 1100, in various examples, can include various types of implantable medical devices, such as, but not limited to, a cardiac implantable pulse generator, a pacemaker, an implantable defibrillator, a neurostimulator, a cardiac monitor, a cochlear implant, etc. Although the implantable medical devices 100, 700, 1100 of FIGS. 1, 7, and 11 are configured to be implanted within a chest of the patient, in various examples, the implantable medical devices 100, 700, 1100 can be implanted at various locations within the patient, including, but not limited to, a chest, a skull, a limb, a back, a pelvic region, etc. of the patient.

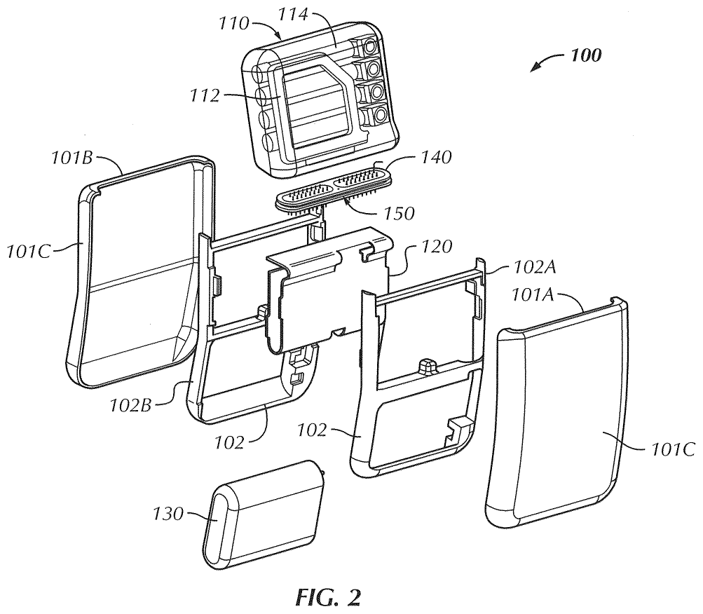

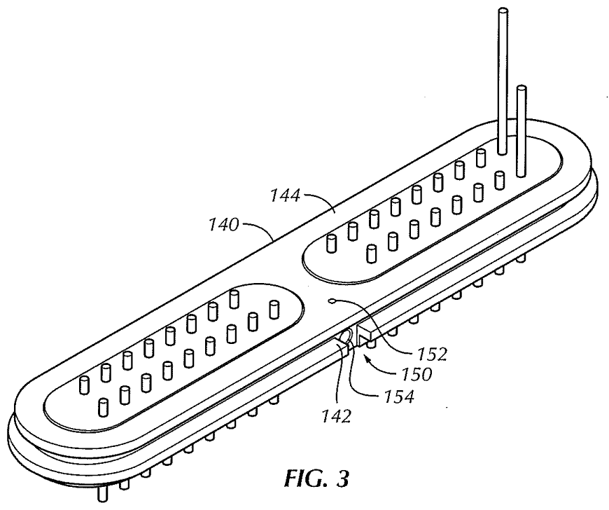

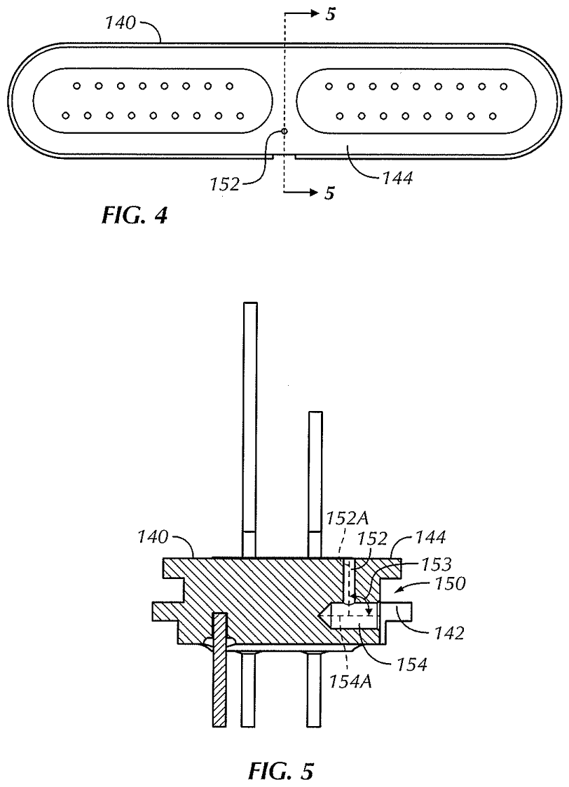

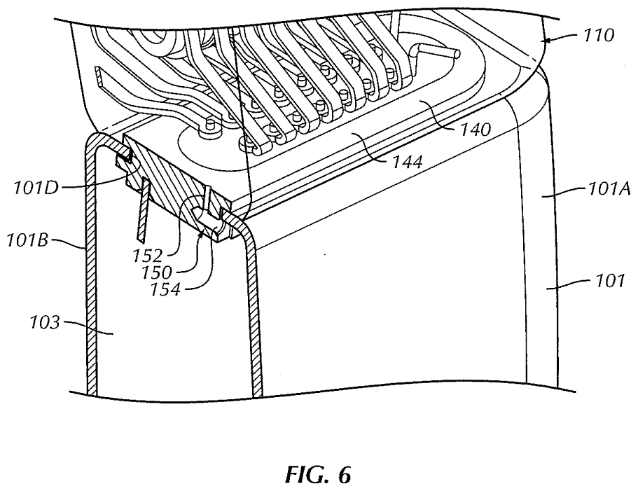

Referring now to FIGS. 1-6, in some examples, the implantable medical device 100 includes a header 110 mounted on an enclosure 101. The header 110, in some examples, includes one or more components disposed within the header 110. In some examples, the components include one or more coils or antennas 112 for communication and/or charging of the implantable medical device 100, one or more connector assemblies 114 for receiving and electrically coupling to one or more leads (not shown), one or more electrical connectors, etc., or a combination thereof. In some examples, the header 110 includes a material disposed around the one or more components disposed within the header 110. In some examples, the header 110 includes epoxy molded around the one or more components disposed within the header 110.

It is contemplated that the enclosure 101, in some examples, is formed from a metallic material, such as titanium or a titanium alloy, for instance, although other metallic materials are contemplated. In various examples, the enclosure 101 can be deep drawn, shallow drawn, stamped, machined, or the like or a combination thereof. In some examples, the enclosure 101 includes a first enclosure portion 101A and a second enclosure portion 101B joined together to form the enclosure 101. In some examples, the enclosure 101 includes an enclosure wall 101C surrounding and defining an interior 103 within the enclosure 101. In some examples, the enclosure 101 includes an opening 101D within the enclosure wall 101C. In some examples, a feedthrough 140 is sealingly disposed within the opening 101D of the enclosure wall 101C. With the first enclosure portion 101A and the second enclosure portion 101B joined and sealed together and the feedthrough 140 disposed and sealed within the opening 101D of the enclosure 101, in some examples, the interior 103 of the enclosure 101 is sealed and not in fluid communication with an exterior of the enclosure 101. In some examples, the interior 103 of the enclosure 101 is hermetically sealed.

Within the enclosure 101, in some examples, are various inner components of the implantable medical device 100. The types of inner components vary with the type of the implantable medical device 100. For illustrative purposes, in some examples, the inner components of the implantable medical device 100 shown in FIGS. 1-6 include electronic components 120 and a battery 130. In various examples, the electronic components 120 can include a control module, a communication module, a charging module, an analysis module, or the like, or a combination thereof. However, this is not intended to be limiting. As such, in other examples, the inner components of the implantable medical device 100 can include more or fewer other and/or different components than those shown and described herein. In some examples, the inner components of the implantable medical device 100 can be anchored, positioned, or otherwise disposed within a frame 102 disposed within the enclosure 101. In some examples, the frame 102 maintains the inner components fixed and properly located within the enclosure 101 to help avoid jostling, damage, and/or improper positioning of the inner components within the enclosure 101 of the implantable medical device 100. In some examples, the frame 102 includes a first frame portion 102A and a second frame portion 102B that can be slid into place on either side of the inner components to essentially sandwich the inner components in between the first and second frame portions 102A, 102B. In other examples, however, the frame 102 can be formed from a single unitary piece. The frame 102, in some examples, can be formed from a polymeric material. In further examples, the frame 102 can be formed from a plastic material.

In some examples, the implantable medical device 100 includes a backfill feature 150. The backfill feature 150, in some examples, allows for temporary fluid access from the exterior of the implantable medical device 100 to the interior 103 of the enclosure 101 once the enclosure 101 and the feedthrough 140 are joined together. In some examples, the backfill feature 150 is disposed within the feedthrough 140. In further examples, the backfill feature 150 is integrated within a flange 142 of the feedthrough 140. In some examples, the backfill feature 150 allows for backfilling of the interior 103 with a fluid and then sealing of the backfill feature 150 to then fluidly isolate the interior 103 of the enclosure 101 from the exterior of the implantable medical device 100, thereby capturing the fluid that was backfilled within the interior 103 of the enclosure 101. In some examples, the fluid can include a gas and/or a liquid, depending on the requirements and application of the implantable medical device 100. In some examples, the fluid is a gas that includes helium. In some examples, the fluid provides an inert atmosphere within the interior 103 of the enclosure 101 to reduce the possibility of any deterioration or corrosion of the inner components within the interior 103 of the enclosure 101.

In some examples, the backfill feature 150 includes a backfilling channel 152 open to the exterior of the implantable medical device 100. The backfilling channel 152, in some examples, includes a longitudinal axis 152A. In some examples, the backfilling channel 152 extends into the feedthrough 140 from a top surface 144 of the feedthrough 140. In this way, in some examples, since the header 110, once in place, covers over the top surface 144 of the feedthrough 140, the backfilling channel 152 is accessible and open to the exterior of the implantable medical device 100 prior to attachment of the header 110 to the enclosure 101. In turn, in some examples, once the header 110 is attached to the enclosure 101, the backfilling channel 152 of the backfill feature 150 is covered by the header 110.

In some examples, the backfill feature 150 includes a welding protection channel 154 fluidly coupled to the backfilling channel 152 and extending from the backfilling channel 152 to the interior 103 of the enclosure 101. In some examples, the welding protection channel 154 includes a longitudinal axis 154A. In some examples, the backfilling channel 152 and the welding protection channel 154 fluidly couple the exterior of the implantable medical device 100 and the interior 103 of the enclosure 101 to allow for fluid backfilling of the interior 103 of the enclosure 101. The backfilling channel 152, in some examples, is configured to be sealed with welding of the backfilling channel 152 after fluid backfilling of the interior 103 of the enclosure 101. That is, in some examples, once the interior 103 of the enclosure 101 has been filled with fluid via the backfill feature 150, a portion of the backfilling channel 152 can be welded shut to effectively close off the backfilling channel 152 and seal the interior 103 of the enclosure 101. In some examples, the backfilling channel 152 can be laser welded to seal off the backfilling channel 152. In this way, in some examples, once the backfilling channel 152 is sealed, the interior 103 of the enclosure 101 is completely sealed with the fluid being captured within the interior 103 of the enclosure 101. In some examples, with the enclosure 101 completely sealed, the interior 103 of the enclosure 101 includes a hermetic environment.

In some examples, the welding protection channel 154 is configured to catch any metallic projections or other debris resulting from the welding of the backfilling channel 152, thereby inhibiting the metallic projections or other debris from entering the interior 103 of the enclosure 101. In some examples, the welding protection channel 154 is disposed at an angle 153 to the backfilling channel 152. The angle 153, in some examples, is measured between the axis 152A of the backfilling channel 152 and the axis 154A of the welding protection channel 154. In some examples, the angle 153 between the backfilling channel 152 and the welding protection channel 152 is less than 180 degrees and greater than zero degrees. It other examples, the angle 153 between the backfilling channel 152 and the welding protection channel 154 is less than 135 degrees and greater than 45 degrees. In still other examples, the angle 153 between the backfilling channel 152 and the welding protection channel 154 is about 90 degrees. Moreover, in some examples, the backfilling channel 152 includes a cross section that is smaller than a cross section of the welding protection channel 154. In some examples, the backfilling channel 152 is substantially circular in cross section and the welding protection channel 154 is substantially circular in cross section, with a diameter of the backfilling channel 152 being smaller than a diameter of the welding protection channel 154. In other examples, one or both of the backfilling channel 152 and the welding protection channel 154 can have a cross section other than circular (such as, for instance, elliptical, rectangular, triangular, etc.) with a width of the backfilling channel 152 being smaller than a width of the welding protection channel 154. The larger cross section of the weld protection channel 154, in some examples, allows for greater surface area of the welding protection channel 154 to better catch the metal projections or other debris resulting from the welding process.

In this way, any metal projections or other debris resulting from the welding process travels generally through the backfilling channel 152 and enters the welding protection channel 154. Since, in some examples, the welding protection channel 154 is at the angle 153 to the backfilling channel 152, the metal projections or other debris continue along their path, traveling across the welding protection channel 154 and hitting a side of the welding protection channel 154 across from the backfilling channel 152. The metal projections or other debris, in some examples, tend to stick to the side of the welding protection channel 154, thereby reducing the chance that a metal projection or other piece of debris actually enters the interior 103 of the enclosure 101 and potentially damages any inner components of the implantable medical device 100. In some examples, by having the backfilling channel 152 and the welding protection channel 154 perpendicularly oriented with respect to one another, when welding shut the smaller backfilling channel 152, any metallic projections or other debris that could be projected inside of the implantable medical device 100 during the welding process will likely fall into the larger cavity of the welding protection channel 154 and, therefore, help avoid any damage to the inner components of the implantable medical device 100.

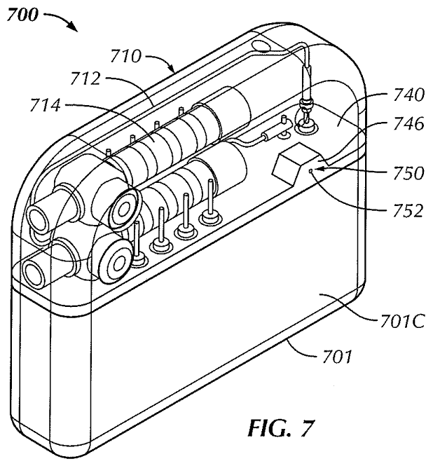

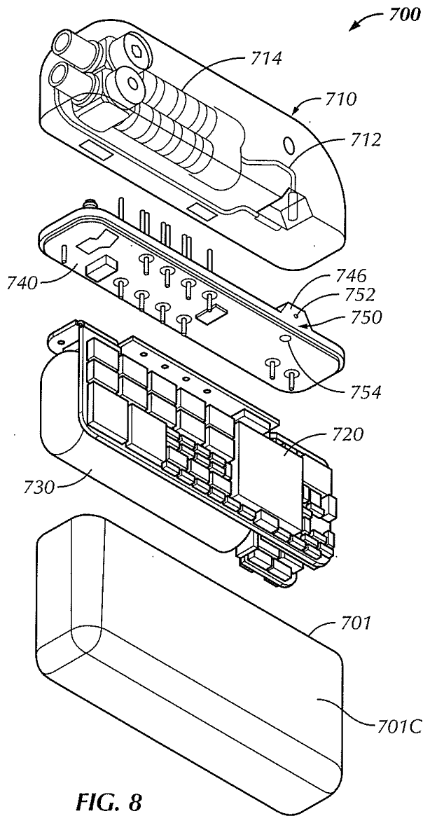

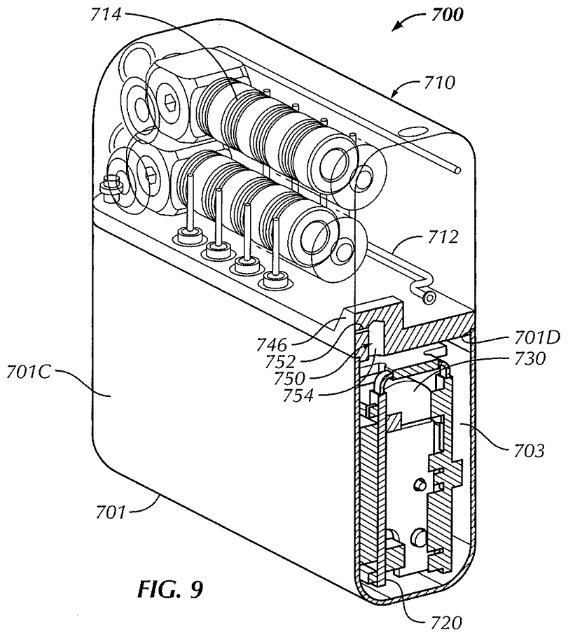

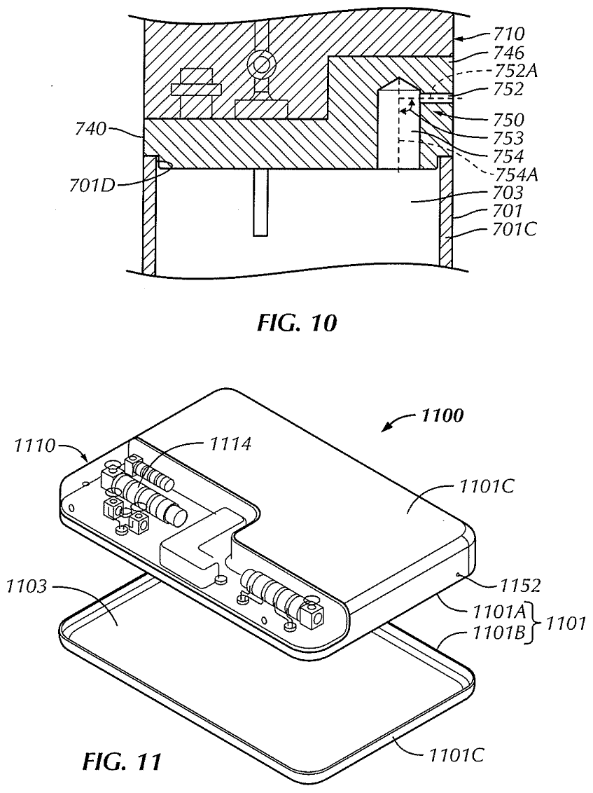

Referring now to FIGS. 7-10, in some examples, the implantable medical device 700 includes a header 710 mounted on an enclosure 701. The header 710, in some examples, includes one or more components disposed within the header 710. In some examples, the components include one or more coils or antennas 712 for communication and/or charging of the implantable medical device 700, one or more connector assemblies 714 for receiving and electrically coupling to one or more leads (not shown), one or more electrical connectors, etc., or a combination thereof. In some examples, the header 710 includes a material disposed around the one or more components disposed within the header 710. In some examples, the header 710 includes epoxy molded around the one or more components disposed within the header 710.

It is contemplated that the enclosure 701, in some examples, is formed from a metallic material, such as titanium or a titanium alloy, for instance, although other metallic materials are contemplated. In various examples, the enclosure 701 can be deep drawn, shallow drawn, stamped, machined, or the like or a combination thereof. In some examples, the enclosure 701 includes an enclosure wall 701C surrounding and defining an interior 703 within the enclosure 701. In some examples, the enclosure 701 includes an opening 701D within the enclosure wall 701C. In some examples, a feedthrough 740 is sealingly disposed within the opening 701D of the enclosure wall 701C. With the feedthrough 740 disposed and sealed within the opening 701D of the enclosure 701, in some examples, the interior 703 of the enclosure 701 is sealed and not in fluid communication with an exterior of the enclosure 701. In some examples, the interior 703 of the enclosure 701 is hermetically sealed.

Within the enclosure 701, in some examples, are various inner components of the implantable medical device 700. The types of inner components vary with the type of the implantable medical device 700. For illustrative purposes, in some examples, the inner components of the implantable medical device 700 shown in FIGS. 7-10 include electronic components 720 and a battery 730. In various examples, the electronic components 720 can include a control module, a communication module, a charging module, an analysis module, or the like, or a combination thereof. However, this is not intended to be limiting. As such, in other examples, the inner components of the implantable medical device 700 can include more or fewer other and/or different components than those shown and described herein.

In some examples, the implantable medical device 700 includes a backfill feature 750. The backfill feature 750, in some examples, allows for temporary fluid access from the exterior of the implantable medical device 700 to the interior 703 of the enclosure 701 once the enclosure 701 and the feedthrough 740 are joined together. In some examples, the backfill feature 750 is disposed within the feedthrough 740. In further examples, the backfill feature 750 is integrated within a protrusion 746 or other feature of the feedthrough 740. In some examples, the backfill feature 750 allows for backfilling of the interior 703 with a fluid and then sealing of the backfill feature 750 to then fluidly isolate the interior 703 of the enclosure 701 from the exterior of the implantable medical device 700, thereby capturing the fluid that was backfilled within the interior 703 of the enclosure 701. In some examples, the fluid can include a gas and/or a liquid, depending on the requirements and application of the implantable medical device 700. In some examples, the fluid is a gas that includes helium. In some examples, the fluid provides an inert atmosphere within the interior 703 of the enclosure 701 to reduce the possibility of any deterioration or corrosion of the inner components within the interior 703 of the enclosure 701.

In some examples, the backfill feature 750 includes a backfilling channel 752 open to the exterior of the implantable medical device 700. The backfilling channel 752, in some examples, includes a longitudinal axis 752A. In some examples, the backfilling channel 752 extends into the feedthrough 740 from the protrusion 746 of the feedthrough 740. By placing the backfilling channel 752 within the protrusion 746 of the feedthrough 740, in some examples, the backfilling channel 752 can be accessible even once the header 710 is in place and attached to the enclosure 701. In some examples, the header 710 can be formed around the protrusion 746 of the feedthrough 710 such that the backfill feature is accessible from the exterior of the implantable medical device 700 with the header 710 attached to the enclosure 701. In this way, in some examples, the position and geometry of the backfilling channel 752 allows for the backfilling channel 752 to not be covered with epoxy after the header 710 is formed on the implantable medical device 700, enabling the header 710 to be made in parallel to the rest of the implantable medical device 700. This allows backfilling of the implantable medical device 700 with a fluid (such as, but not limited to an inert gas) after the header 710 is welded to the enclosure 701. In some examples, the design of the feedthrough 740 with the backfilling channel 752 disposed within the protrusion 746 allows for the interior 703 of the enclosure 701 to be backfilled after the epoxy of the header 710 is formed.

In some examples, the backfill feature 750 includes a welding protection channel 754 fluidly coupled to the backfilling channel 752 and extending from the backfilling channel 752 to the interior 703 of the enclosure 701. In some examples, the welding protection channel 754 includes a longitudinal axis 754A. In some examples, the backfilling channel 752 and the welding protection channel 754 fluidly couple the exterior of the implantable medical device 700 and the interior 703 of the enclosure 701 to allow for fluid backfilling of the interior 703 of the enclosure 701. The backfilling channel 752, in some examples, is configured to be sealed with welding of the backfilling channel 752 after fluid backfilling of the interior 703 of the enclosure 101. That is, in some examples, once the interior 703 of the enclosure 701 has been filled with fluid via the backfill feature 750, a portion of the backfilling channel 752 can be welded shut to effectively close off the backfilling channel 752 and seal the interior 703 of the enclosure 701. In some examples, the backfilling channel 752 can be laser welded to seal off the backfilling channel 752. In this way, in some examples, once the backfilling channel 752 is sealed, the interior 703 of the enclosure 701 is completely sealed with the fluid being captured within the interior 703 of the enclosure 701. In some examples, with the enclosure 701 completely sealed, the interior 703 of the enclosure 701 includes a hermetic environment.

In some examples, the welding protection channel 754 is configured to catch any metallic projections or other debris resulting from the welding of the backfilling channel 752, thereby inhibiting the metallic projections or other debris from entering the interior 703 of the enclosure 701. In some examples, the welding protection channel 754 is disposed at an angle 753 to the backfilling channel 752. The angle 753, in some examples, is measured between the axis 752A of the backfilling channel 752 and the axis 754A of the welding protection channel 754. In some examples, the angle 753 between the backfilling channel 752 and the welding protection channel 752 is less than 180 degrees and greater than zero degrees. It other examples, the angle 753 between the backfilling channel 752 and the welding protection channel 754 is less than 135 degrees and greater than 45 degrees. In still other examples, the angle 753 between the backfilling channel 752 and the welding protection channel 754 is about 90 degrees. Moreover, in some examples, the backfilling channel 752 includes a cross section that is smaller than a cross section of the welding protection channel 754. In some examples, the backfilling channel 752 is substantially circular in cross section and the welding protection channel 754 is substantially circular in cross section, with a diameter of the backfilling channel 752 being smaller than a diameter of the welding protection channel 754. In other examples, one or both of the backfilling channel 752 and the welding protection channel 754 can have a cross section other than circular (such as, for instance, elliptical, rectangular, triangular, etc.) with a width of the backfilling channel 752 being smaller than a width of the welding protection channel 754. The larger cross section of the weld protection channel 754, in some examples, allows for greater surface area of the welding protection channel 754 to better catch the metal projections or other debris resulting from the welding process.

In this way, any metal projections or other debris resulting from the welding process travels generally through the backfilling channel 752 and enters the welding protection channel 754. Since, in some examples, the welding protection channel 754 is at the angle 753 to the backfilling channel 752, the metal projections or other debris continue along their path, traveling across the welding protection channel 754 and hitting a side of the welding protection channel 754 across from the backfilling channel 752. The metal projections or other debris, in some examples, tend to stick to the side of the welding protection channel 754, thereby reducing the chance that a metal projection or other piece of debris actually enters the interior 703 of the enclosure 701 and potentially damages any inner components of the implantable medical device 700. In some examples, by having the backfilling channel 752 and the welding protection channel 754 perpendicularly oriented with respect to one another, when welding shut the smaller backfilling channel 752, any metallic projections or other debris that could be projected inside of the implantable medical device 700 during the welding process will likely fall into the larger cavity of the welding protection channel 754 and, therefore, help avoid any damage to the inner components of the implantable medical device 700.

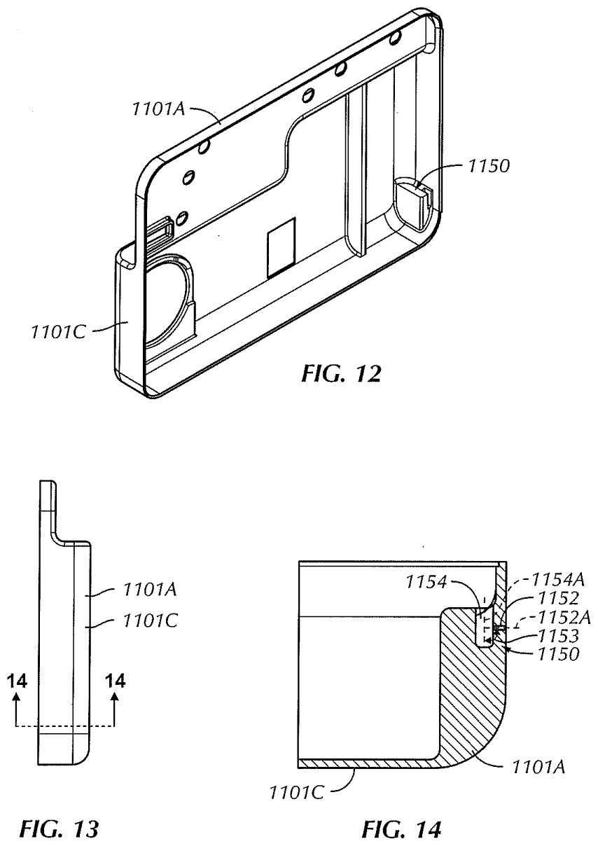

Referring now to FIGS. 11-14, in some examples, the implantable medical device 1100 includes a header 1110 mounted on an enclosure 1101. The header 1110, in some examples, includes one or more components disposed within the header 1110. In some examples, the components include one or more coils or antennas for communication and/or charging of the implantable medical device 1100, one or more connector assemblies 1114 for receiving and electrically coupling to one or more leads (not shown), one or more electrical connectors, etc., or a combination thereof. In some examples, the header 1110 includes a material disposed around the one or more components disposed within the header 1110. In some examples, the header 1110 includes epoxy molded around the one or more components disposed within the header 1110.

It is contemplated that the enclosure 1101, in some examples, is formed from a metallic material, such as titanium or a titanium alloy, for instance, although other metallic materials are contemplated. In various examples, the enclosure 1101 can be deep drawn, shallow drawn, stamped, machined, or the like or a combination thereof. In some examples, the enclosure 1101 includes a first enclosure portion 1101A and a second enclosure portion 1101B joined together to form the enclosure 1101. In some examples, the enclosure 1101 includes an enclosure wall 1101C surrounding and defining an interior 1103 within the enclosure 1101. With the first enclosure portion 1101A and the second enclosure portion 1101B joined and sealed together, in some examples, the interior 1103 of the enclosure 1101 is sealed and not in fluid communication with an exterior of the enclosure 1101. In some examples, the interior 1103 of the enclosure 1101 is hermetically sealed.

Within the enclosure 1101, in some examples, are various inner components of the implantable medical device 1100. The types of inner components vary with the type of the implantable medical device 1100. In some examples, the inner components of the implantable medical device 1100 can include various components including, but not limited to, one or more of electronic components, a battery, a control module, a communication module, a charging module, an analysis module, or the like, or a combination thereof. However, this is not intended to be limiting. As such, in other examples, the inner components of the implantable medical device 1100 can include more or fewer other and/or different components than those described herein.

In some examples, the implantable medical device 1100 includes a backfill feature 1150. The backfill feature 1150, in some examples, allows for temporary fluid access from the exterior of the implantable medical device 1100 to the interior 1103 of the enclosure 1101 once the first and second enclosure portions 1101A, 1101B are joined together. In some examples, the backfill feature 1150 is disposed within the enclosure wall 1101C of the enclosure 1101. In some examples, the backfill feature 1150 allows for backfilling of the interior 1103 with a fluid and then sealing of the backfill feature 1150 to then fluidly isolate the interior 1103 of the enclosure 1101 from the exterior of the implantable medical device 1100, thereby capturing the fluid that was backfilled within the interior 1103 of the enclosure 1101. In some examples, the fluid can include a gas and/or a liquid, depending on the requirements and application of the implantable medical device 1100. In some examples, the fluid is a gas that includes helium. In some examples, the fluid provides an inert atmosphere within the interior 1103 of the enclosure 1101 to reduce the possibility of any deterioration or corrosion of the inner components within the interior 1103 of the enclosure 1101.

In some examples, the backfill feature 1150 includes a backfilling channel 1152 open to the exterior of the implantable medical device 1100. The backfilling channel 1152, in some examples, includes a longitudinal axis 1152A. In some examples, the backfill feature 1150 is disposed within the enclosure wall 1101C. In some examples, the backfilling channel 1152 extends into the enclosure wall 1101C from an exterior surface of the enclosure 1101. By placing the backfilling channel 1152 within the enclosure wall 1101C in a location away from the header 1110, in some examples, the backfilling channel 1152 can be accessible even once the header 1110 is in place and attached to the enclosure 1101. In this way, in some examples, the position and geometry of the backfilling channel 1152 allows for the backfilling channel 1152 to not be covered with epoxy after the header 1110 is formed on the implantable medical device 1100, enabling the header 1110 to be made in parallel to the rest of the implantable medical device 1100. This allows backfilling of the implantable medical device 1100 with a fluid (such as, but not limited to an inert gas) after the header 1110 is welded to the enclosure 1101. In some examples, the placement of the backfilling channel 1152 within the enclosure wall 1101C allows for the interior 1103 of the enclosure 1101 to be backfilled after the epoxy of the header 1110 is formed. In some examples, the location of the backfill feature 1150 within the enclosure wall 1101C allows for the backfilling process to be performed after the epoxy casting process of the header 1110 in the implantable medical device 1100 with a shallow-drawn enclosure 1101.

In some examples, the backfill feature 1150 is integrally formed within at least one of the first enclosure portion 1101A and the second enclosure portion 1101B. In further examples, the backfill feature 1150 is machined within at least one of the first enclosure portion 1101A and the second enclosure portion 1101B. In some examples, the backfill feature 1150 is configured to protect the interior 1103 of the implantable medical device 1100 during welding of the backfilling channel 1152 of the enclosure 1101 of the implantable medical device 1100. In some examples, the enclosure 1101 of the implantable medical device 1100 is backfilled with helium or another inert gas, for instance, to test the enclosure 1101 for hermeticity. In such cases, the backfilling channel 1152 can be used to fill the enclosure 1101 with helium or the like. After filling, the backfilling channel 1152 can be welded closed to seal the enclosure 1101. In some examples, the backfilling channel 1152 is welded using a laser beam. This weld can generate some metallic projections. Containment of such metallic projections and the laser beam is important in order to minimize, if not avoid, damage to the inner components such as electronics within the enclosure 1101 during welding.

In some examples, the backfill feature 1150 includes a welding protection channel 1154 fluidly coupled to the backfilling channel 1152 and extending from the backfilling channel 1152 to the interior 1103 of the enclosure 1101. In some examples, the welding protection channel 1154 includes a longitudinal axis 1154A. In some examples, the backfilling channel 1152 and the welding protection channel 1154 fluidly couple the exterior of the implantable medical device 1100 and the interior 1103 of the enclosure 1101 to allow for fluid backfilling of the interior 1103 of the enclosure 1101. The backfilling channel 1152, in some examples, is configured to be sealed with welding of the backfilling channel 1152 after fluid backfilling of the interior 1103 of the enclosure 1101. That is, in some examples, once the interior 1103 of the enclosure 1101 has been filled with fluid via the backfill feature 1150, a portion of the backfilling channel 1152 can be welded shut to effectively close off the backfilling channel 1152 and seal the interior 1103 of the enclosure 1101. In some examples, the backfilling channel 1152 can be laser welded to seal off the backfilling channel 1152. In this way, in some examples, once the backfilling channel 1152 is sealed, the interior 1103 of the enclosure 1101 is completely sealed with the fluid being captured within the interior 1103 of the enclosure 1101. In some examples, with the enclosure 1101 completely sealed, the interior 1103 of the enclosure 1101 includes a hermetic environment.

In some examples, the welding protection channel 1154 is configured to catch any metallic projections or other debris resulting from the welding of the backfilling channel 1152, thereby inhibiting the metallic projections or other debris from entering the interior 1103 of the enclosure 1101. In some examples, the welding protection channel 1154 is disposed at an angle 1153 to the backfilling channel 1152. The angle 1153, in some examples, is measured between the axis 1152A of the backfilling channel 1152 and the axis 1154A of the welding protection channel 1154. In some examples, the angle 1153 between the backfilling channel 1152 and the welding protection channel 1152 is less than 180 degrees and greater than zero degrees. It other examples, the angle 1153 between the backfilling channel 1152 and the welding protection channel 1154 is less than 135 degrees and greater than 45 degrees. In still other examples, the angle 1153 between the backfilling channel 1152 and the welding protection channel 1154 is about 90 degrees. Moreover, in some examples, the backfilling channel 1152 includes a cross section that is smaller than a cross section of the welding protection channel 1154. In some examples, the backfilling channel 1152 is substantially circular in cross section and the welding protection channel 1154 is substantially circular in cross section, with a diameter of the backfilling channel 1152 being smaller than a diameter of the welding protection channel 1154. In other examples, one or both of the backfilling channel 1152 and the welding protection channel 1154 can have a cross section other than circular (such as, for instance, elliptical, rectangular, triangular, etc.) with a width of the backfilling channel 1152 being smaller than a width of the welding protection channel 1154. The larger cross section of the weld protection channel 1154, in some examples, allows for greater surface area of the welding protection channel 1154 to better catch the metal projections or other debris resulting from the welding process.

In this way, any metal projections or other debris resulting from the welding process travels generally through the backfilling channel 1152 and enters the welding protection channel 1154. Since, in some examples, the welding protection channel 1154 is at the angle 1153 to the backfilling channel 1152, the metal projections or other debris continue along their path, traveling across the welding protection channel 1154 and hitting a side of the welding protection channel 1154 across from the backfilling channel 1152. The metal projections or other debris, in some examples, tend to stick to the side of the welding protection channel 1154, thereby reducing the chance that a metal projection or other piece of debris actually enters the interior 1103 of the enclosure 1101 and potentially damages any inner components of the implantable medical device 1100. In some examples, by having the backfilling channel 1152 and the welding protection channel 1154 perpendicularly oriented with respect to one another, when welding shut the smaller backfilling channel 1152, any metallic projections or other debris that could be projected inside of the implantable medical device 1100 during the welding process will likely fall into the larger cavity of the welding protection channel 1154 and, therefore, help avoid any damage to the inner components of the implantable medical device 1100.

For welding protection, typically, a titanium strip is used to protect the inner electrical components from any metallic ejections that may occur during the welding process. This strip is often welded to the enclosure or the lid of the device or secured to the assembly by a plastic nest. In some examples, by integrally incorporating the backfill feature 1150 within the enclosure 1101 (for instance, by machining the backfill feature 1150 within the enclosure 1101 itself), the need for any such separate strip to be attached to the enclosure at a later stage is eliminated. In this way, the steps needed to assemble the implantable medical device 1100 are reduced, which allows for a more repeatable process by which to provide protection of one or more of the internal components within the enclosure 1101 during welding procedures.

The present inventors have recognized various advantages of the subject matter described herein. The present inventors have recognized, among other things, that the present inventive subject matter can be used to reduce a number of components needed in implantable medical devices, thereby reducing the complexity of the assembly, reducing assembly times, and allowing for a smaller device by reducing the need for a weld protection band to protect inner electrical components during welding of the device. In various examples, the present inventive subject matter is advantageous in that it provides a machined backfilling hole that, in some examples of the present inventive subject matter, can be better controlled in dimensions and tolerances. Moreover, in some examples, the present inventive subject matter provides for a header assembly to be made in parallel with the rest of the device assembly, thereby reducing the assembly times. Also, by casting the header assembly without the hermetic enclosure assembly, the subassembly volume for each unit is reduced and more units can be cast on a single batch, and the curing schedule can be optimized without any limitation/constraint caused by the electronic components (for instance, temperature limitations/constraints). In this way, in various examples, the present inventive subject matter is advantageous in that it provides a significant cost reduction or optimization of components and resources and a process suitable for automatization. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. An implantable medical device comprising:
an enclosure including an enclosure wall, the enclosure wall surrounding and defining an interior within the enclosure, the enclosure including an opening within the enclosure wall;
a feedthrough sealingly disposed within the opening of the enclosure wall; and
a backfill feature including:
a backfilling channel open to an exterior of the implantable medical device; and
a welding protection channel fluidly coupled to the backfilling channel and extending from the backfilling channel to the interior of the enclosure, the welding protection channel being disposed at an angle to the backfilling channel, wherein the backfilling channel and the welding protection channel fluidly couple the exterior of the implantable medical device and the interior of the enclosure to allow for fluid backfilling of the interior of the enclosure, the backfilling channel configured to be sealed with welding of the backfilling channel after fluid backfilling of the interior of the enclosure, the welding protection channel being configured to catch metallic projections from the welding of the backfilling channel to inhibit the metallic projections from entering the interior of the enclosure.

2. The implantable medical device of claim 1, wherein the backfill feature is disposed within the feedthrough.

3. The implantable medical device of claim 2, wherein the backfill feature is integrated within a flange of the feedthrough.

4. The implantable medical device of claim 1, wherein the backfill feature is disposed within the enclosure wall.

5. The implantable medical device of claim 1, wherein, with the feedthrough sealed within the opening of the enclosure and the backfilling channel sealed, the interior of the enclosure includes a hermetic environment.

6. The implantable medical device of claim 1, comprising a header attached to the enclosure, wherein the backfill feature is accessible from the exterior of the implantable medical device with the header attached to the enclosure.

7. The implantable medical device of claim 1, comprising a header attached to the enclosure, wherein the backfill feature is covered by the header with the header attached to the enclosure.

8. The implantable medical device of claim 1, wherein the backfilling channel includes a cross section that is smaller than a cross section of the welding protection channel.

9. The implantable medical device of claim 1, wherein the backfilling channel is circular in cross section and the welding protection channel is circular in cross section, a diameter of the backfilling channel being smaller than a diameter of the welding protection channel.

10. The implantable medical device of claim 1, wherein the angle between the backfilling channel and the welding protection channel is less than 180 degrees and greater than zero degrees.

11. The implantable medical device of claim 1, wherein the angle between the backfilling channel and the welding protection channel is about 90 degrees.

12. An implantable medical device comprising:
an enclosure including an enclosure wall, the enclosure wall surrounding and defining an interior within the enclosure, the enclosure including an opening within the enclosure wall;
a feedthrough sealingly disposed within the opening of the enclosure wall; and
a backfill feature including:
a backfilling channel open to an exterior of the implantable medical device; and
a welding protection channel fluidly coupled to the backfilling channel and extending from the backfilling channel to the interior of the enclosure, the welding protection channel being disposed at an angle to the backfilling channel, the angle being less than 135 degrees and greater than 45 degrees, wherein the backfilling channel includes a cross section that is smaller than a cross section of the welding protection channel, the backfilling channel and the welding protection channel fluidly coupling the exterior of the implantable medical device and the interior of the enclosure to allow for fluid backfilling of the interior of the enclosure, the backfilling channel configured to be sealed with welding of the backfilling channel after fluid backfilling of the interior of the enclosure, the welding protection channel being configured to catch metallic projections from the welding of the backfilling channel to inhibit the metallic projections from entering the interior of the enclosure.

13. The implantable medical device of claim 12, wherein the backfill feature is disposed within the feedthrough.

14. The implantable medical device of claim 13, wherein the backfill feature is integrated within a flange of the feedthrough.

15. The implantable medical device of claim 12, wherein the backfill feature is disposed within the enclosure wall.

16. The implantable medical device of claim 12, wherein, with the feedthrough sealed within the opening of the enclosure and the backfilling channel sealed, the interior of the enclosure includes a hermetic environment.

17. The implantable medical device of claim 12, comprising a header attached to the enclosure, wherein the backfill feature is accessible from the exterior of the implantable medical device with the header attached to the enclosure.

18. The implantable medical device of claim 12, comprising a header attached to the enclosure, wherein the backfill feature is covered by the header with the header attached to the enclosure.

19. The implantable medical device of claim 12, wherein the backfilling channel is circular in cross section and the welding protection channel is circular in cross section, a diameter of the backfilling channel being smaller than a diameter of the welding protection channel.

20. The implantable medical device of claim 12, wherein the angle between the backfilling channel and the welding protection channel is about 90 degrees.
